(19) United States
(12) Patent Application Publication
(10) Pub. No.: US 2019/0125794 A1
(43) Pub. Date: May 2, 2019

(54) NOVEL OPTHALMIC STEM CELL TREATMENT AND METHOD

(71) Applicant: Alimorad Farshchian, North Miami, FL (US)

(72) Inventor: Alimorad Farshchian, North Miami, FL (US)

(21) Appl. No.: 16/172,216

(22) Filed: Oct. 26, 2018

Related U.S. Application Data
(60) Provisional application No. 62/577,521, filed on Oct. 26, 2017.

(51) Int. Cl. A61K 35/12; A61K 47/02; A61K 9/00; A61P 27/02

(52) U.S. Cl. CPC A61K 35/12; A61P 27/02; A61K 9/0048; A61K 47/02

(57) ABSTRACT

A new composition and method for providing ophthalmic stem cell therapy is provided whereby a composition of sodium bicarbonate and stem cells in a 1:1 ratio is administered as an eye drop to a patient and results in measured visual acuity.

### INDEX TO RELATED APPLICATIONS

This application is non-provisional of and claims benefit to U.S. Provisional Patent Application Ser. No. 62/577,521 filed Oct. 26, 2017 the disclosure of which is incorporated by reference in its entirety.

### BACKGROUND OF THE INVENTION

Advances in stem cell therapy are progressing and giving hope to many in need of medical treatment for various ailments. One area that has been significantly difficult has been administering stem cell therapy to the eyes. The unique anatomical and physiological features of the human eye make it particularly troublesome and difficult to effectively administer therapy.

The present invention recognizes this difficulty and has preliminary data proving successful administration for treatment of ophthalmic disorders using stem cell therapy.

### SUMMARY OF THE INVENTION

An effective ophthalmic stem cell composition is provided in the present invention.

In one embodiment, the present invention is a method for providing ophthalmic stem cell therapy, said method comprising the steps of:

preparing a treatment solution of sodium bicarbonate buffer solution and stem cells in a 1:1 ratio;

measuring a first visual acuity measurement prior to administration;

administering 4 cc of said treatment solution as an eye drop into each eye of a patient;

measuring a second visual acuity measurement 24 hours after said administering;

measuring a third visual acuity measurement between 7-10 days after said first visual acuity; said method resulting in an improvement of visual acuity from said first visual acuity measurement to said third visual acuity measurement of two increments for nine months after said administering.

In one embodiment, the method further includes maintaining said third visual acuity measurement improvement of two increments for up to nine months.

The sodium bicarbonate buffer solution is 8.4% w/v.

In one embodiment the method consists of

preparing a treatment solution of sodium bicarbonate buffer solution and stem cells in a 1:1 ratio;

measuring a first visual acuity measurement prior to administration;

administering 4 cc of said treatment solution as an eye drop into each eye of a patient;

measuring a second visual acuity measurement 24 hours after said administering;

measuring a third visual acuity measurement between 7-10 days after said first visual acuity; said method resulting in an improvement of visual acuity from said first visual acuity measurement to said third visual acuity measurement of two increments for nine months after said administering.

In one embodiment, the treatment solution administered as eye drops consists of a treatment solution prepared in a 1:1 ratio of 8.4% w/v of sodium bicarbonate solution:stem cells.

### DETAILED DESCRIPTION OF THE PREFERRED EMBODIMENT

In one embodiment, the present invention is an autologous treatment, which means taking the cells from the patient and administering to the same patient. That is to say, the donor and the recipient are the same person.

In one embodiment, the invention includes a method of using human stem cells and turning it into a component of an eye drop.

Previously, there have been methods whereby the cells are delivered to the eyes by means of injecting stem cells to the eyes to treat the diseases of retina. This was done as an attempt to treat disorders including, but not limited to: diabetes, retinopathy, macular degeneration, as well as retinitis pigmentosa.

The method of the injection although at times working, is inferior to the method of using eye drops because by using the eye drops a practitioner is nourishing the eyes over a period of 24 hours or 48 hours depending on the severity of the disease. In one embodiment, the present invention provides administering eye drops in the eyes every 15 minutes. The production of eye drops in the past has been challenging due to the acidity of the stem cells and the growth factors producing neutral substance to the eye so that it does not burn the eye or disturb or causing irritation to the eye has always been a challenge.

In one embodiment, the method includes stem cells in combination with sodium bicarbonate in order to neutralize the acidity of the eye drops.

Preliminary testing s has resulted in improved visual acuity testing as well as a fundoscopy testing. As is know in the art, Ophthalmoscopy, also called fundoscopy, is a test that allows a health professional to see inside the fundus of the eye and other structures using an ophthalmoscope (or funduscope). It is done as part of an eye examination and may be done as part of a routine physical examination.

Patients, who receive the eye drops, their examination of fundoscopy as well as their visual acuity improves within one week.

The present invention is additionally novel in that the posterior pull of the eye's retina is about 2 inches deep. So, most efforts have focused on injections to reach the region. The present invention is an eye drop that can penetrate deep. Penetrate about 2 inches deep.

Injection of the eye is inferior to this treatment because when you inject the eye, you do it one time. The present invention does not require injecting every 10 minutes as many have attempted. In general, stem cell therapy testing is supported by evidence scientifically that the more it is given, the better it works. It has been speculated that in a eye drop format, the eye drops would need to be delivered every 15 minutes for a treatment period of 2-4 hours.

The solution of the present method, the method, and results demonstrate improvement in a single administration.

In one embodiment, the drops of the present invention are made as follows:

50% stem cells

50% sodium bicarbonate buffer solution 8.4% sodium bicarbonate

Physiological saline is typically pH of 7.4. However, the human eye has a pH of between 7.0-7.3. The eye drops are buffered to a pH of 7.0-7.3.

### Testing and Results

In a clinical trial, 107 patients were tested.

Each patient was administered 4 cc of the solution that is 1:1 (or a 50/50) sodium bicarbonate buffer:stem cells as treatment solution as an eye drop directly into each eye.

Patients were given vision acuity tests before administration, 24 h after administration and at approximately 1 week after administration with the one week testing occurring between 7-10 days after administration.

As is known in the art, Snellen fraction Visual acuity=Distance at which test is made/distance at which the smallest optotype identified subtends an angle of five arcminutes.

Many people know this fraction when referring to “20/20” vision.

The fraction denominator is the number that changes and provides a measure.

As used herein, the term incremental change refers to a change of 5 in the denominator.

Thus, vision that changes from 20/40 to 20/35 indicates an improvement in vision of one increment, 20/40 to 20/30 indicates an improvement of 2 increments, and so on.

It is also important to note that in the study conducted of 107 persons, there were zero adverse events reported.

The results are as follows:

At 24 hours post administration of drops directly into each eye, 101 patients demonstrated vision improvement of 2 increments.

At one week post administration, the same 101 patients maintained vision improvement with no reverting to pre administration acuity. The six remaining patients exhibited vision acuity improvement of 1 increment at the one week time interval.

All patients were examined 3 month, 6 month, 9 nine month, post administration. All patients maintained their improvement in visual acuity improvements through nine months. Although measurements in this study include up to nine months, it is believed that the improved acuity will last up to, at least 5 years.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A method for providing ophthalmic stem cell therapy, said method comprising the steps of:
preparing a treatment solution of sodium bicarbonate buffer solution and stem cells in a 1:1 ratio;
measuring a first visual acuity measurement prior to administration;
administering 4 cc of said treatment solution as an eye drop into each eye of a patient;
measuring a second visual acuity measurement 24 hours after said administering;
measuring a third visual acuity measurement between 7-10 days after said first visual acuity; said method resulting in an improvement of visual acuity from said first visual acuity measurement to said third visual acuity measurement of two increments.

2. The method of claim 1, wherein said method further maintaining said third visual acuity measurement of two increments for up to nine months

3. The method of claim 1, wherein said sodium bicarbonate buffer solution is 8.4% w/v.

4. The treatment solution of claim 1 prepared as an eye drop consisting of sodium bicarbonate buffer solution and stem cells in a 1:1 ratio.
